(19) United States
(12) Patent Application Publication
(10) Pub. No.: US 2015/0005759 A1
(43) Pub. Date: Jan. 1, 2015

(54) Current Delivery Systems, Apparatuses and Methods

(71) Applicants: Richard Shaun Welches, Woburn, MA (US); Daniel Hohm, Merrimac, NH (US)

(72) Inventors: Richard Shaun Welches, Woburn, MA (US); Daniel Hohm, Merrimac, NH (US)

(73) Assignee: CYNOSURE, INC., Westford, MA (US)

(21) Appl. No.: 14/212,054

(22) Filed: Mar. 14, 2014

Related U.S. Application Data
(60) Provisional application No. 61/782,446, filed on Mar. 14, 2013.

(51) Int. Cl. A61B 18/14; H05B 6/04

(52) U.S. Cl. CPC A61B 18/1402; H05B 6/04; A61B 2018/00005
USPC 606/34; 219/660

(57) ABSTRACT

In part, the disclosure relates to an electromagnetic current displacement apparatus that includes one or more magnetic field sources and an alternating current source. The apparatus includes current delivery electrodes that may be part of a cuff, a hand held device, or individual electrode pads suitable for temporary fixation to skin. In one embodiment, an alternating current is transcutaneously delivered using skin contacting electrodes sized and arranged to avoid hotspots and provide a uniform delivery of the current. In turn, current attractors and repulsors can be arranged on the skin or in a suitable device to push or pull sections of the current that is disposed below such elements. Magnetic fields can be applied and focused to the regions through which the current passes, effectively pushing the current deeper into a target region below the surface of the skin.

### RELATED APPLICATION

This application claims priority to U.S. Provisional Patent Application No. 61/782,446 filed on Mar. 14, 2013, the disclosure of which is herein incorporated by reference in its entirety.

### FIELD OF THE INVENTION

In general, the disclosure relates to the field of non-invasive procedures. More specifically, the disclosure relates to systems, apparatuses and methods for delivering a spatially targeted alternating current to a material such as a tissue.

### BACKGROUND OF THE INVENTION

Various treatment and aesthetic procedures have grown in popularity for tissue treatments to promote health and improve appearances. These procedures are based upon various technologies such as lasers, focused ultrasound, selective cryolysis, radiofrequency devices, and electrosurgical devices.

Electrosurgical (“ES”) devices are often used for cutting and other invasive procedures such as those described in United States Patent Applications 20130035688, 20130030429, 20130060250, and 20130006239. The invasive nature of electrosurgical cutters and other types of invasive devices can increase the need for post-treatment visits and necessitate additional treatment regimens such as stiches and antibiotics.

Noninvasive or minimally invasive surgical or aesthetic procedures that enable a quick recovery with few side-effects are always in demand. Unfortunately, various non-invasive procedures such as heating tissue using a water bath or applying surface coolant cannot be reliably controlled to provide the desired effect at a particular depth or region below the skin. Similarly, various electricity-based procedures also lack tissue targeting capabilities. A need therefore exists for non-invasive procedures and other procedures that facilitate targeting of treatment regions below the surface of the skin in a controlled manner with improve recovery times.

### SUMMARY OF INVENTION

In one aspect, the disclosure relates to an electrical energy delivery apparatus. The apparatus includes a control system comprising a user interface; an alternating current source comprising a current output, the alternating current source in electrical communication with the control system; a first electrode in electrical communication with the current output; a second electrode disposed a distance d from the first electrode; and a first magnetic field source comprising a first magnetic field output, the first magnetic field source in electrical communication with the control system, the first magnetic field source disposed between the first electrode and the second electrode.

In one embodiment, the alternating current source is a closed loop current source. In one embodiment, the apparatus further includes a first housing including a first housing surface defining a first opening, wherein a portion of the first electrode spans the first opening. In one embodiment, the apparatus further includes a second housing including a second housing surface defining a second opening, wherein a portion of the second electrode spans the second opening. In one embodiment, the apparatus further includes a housing including a housing surface defining a first opening and a second opening, wherein a portion of the first electrode spans the first opening, wherein a portion of the second electrode spans the second opening.

In one embodiment, the apparatus further includes a second magnetic field source including a second magnetic field output, the second magnetic field source disposed between the first electrode and the second electrode, the second magnetic field source in electrical communication with the control system. In one embodiment, d ranges from about 10 mm to about 100 mm.

In one embodiment, the apparatus further includes a phase monitor in electrical communication with the control system and the second electrode. In one embodiment, the control system includes a feedback loop that receives a first phase value at the second electrode and adjusts a second phase value associated with field inducing current of the magnetic field source. In one embodiment, the current generated from the alternating current source ranges from about 50 mA to about 3 A. In one embodiment, the apparatus further includes a cooler in electrical communication with the control system.

In one embodiment, the system further includes a first driver in electrical communication with the first magnetic field source, the first driver in electrical communication with the control system, wherein the first magnetic field source includes a first coil. In one embodiment, the apparatus further includes a static attractor in electrical communication with the control system.

In one aspect, the disclosure relates to a method of directing electrical energy to one or more locations in a region of tissue below a skin surface. The method includes generating an alternating transcutaneous current that flows between a first electrode and a second electrode to define a first current channel having a first length, the first electrode and the second electrode separated by a distance d and disposed on the skin surface; noninvasively applying one or more magnetic fields to the skin surface that repel the alternating transcutaneous current in a direction opposite that of the skin surface until the alternating transcutaneous current reaches one or more locations and defines a second current channel having a second length, the second length greater than the first length; and heating tissue in a target region that includes a portion of the second current channel disposed between the first electrode and the second electrode.

In one embodiment, the method further includes substantially linearizing the second current path such that the flow of the alternating transcutaneous current occurs along a substantially straight line segment which defines more than 50% of the second length. In one embodiment, the method further includes generating the one or more magnetic fields using an alternating magnetic field inducing current passing through a coil. In one embodiment, the method further includes synchronizing a first phase of the alternating transcutaneous current and a second phase of the alternating magnetic field inducing current such that the first phase and the second phase are substantially the same or offset by a predetermined control phase value. In one embodiment, the one or more magnetic fields are noninvasively applied at an angle measured relative to a normal to the skin surface, wherein the angle ranges from about 5 degrees to less than or equal to about 45 degrees. In one embodiment, one or more magnetic fields are noninvasively applied at an angle measured relative to a normal to the skin surface, wherein the angle ranges from greater than about 0 degrees to less than or equal to about 90 degrees.

In one embodiment, the method further includes cooling the skin surface in a region around the first electrode and the second electrode. In one embodiment, the method further includes moving the alternating transcutaneous current within a tissue region back and forth between the second current path and another current path by periodically changing the applied magnetic field. In one embodiment, the method further includes moving one or more sections of the second current path using one or more static attractors disposed between the first electrode and the second electrode. In one embodiment, the method further includes cooling the tissue such that an impedance change results by which the transcutaneous current moves to another treatment region.

One or more embodiments of the disclosure are directed to a current delivery apparatus such as an electromagnetic current displacement (“EMID”) apparatus with external magnetic field coils and injection current expansion electrodes. Embodiments of the apparatus include injection electrodes for temporary fixation to the patient's skin. According to one embodiment, an energy delivery device generates an injection current that ranges from about 50 mA to about 5 A or more in a target tissue region. In one embodiment, the alternating transcutaneous current in the tissue ranges from about 50 mArms to about 5 Arms. External magnetic fields are applied to the skin surface such that lines of magnetic flux pass through the region in which the current originally flows from one or more electrodes. The magnetic fields effectively push the transcutaneous alternating current deeper below the surface of the skin to a location in the material such as tissue where a non-invasive target treatment is desired. In one embodiment, the alternating magnetic field inducing current used in a magnetic field source is sufficient to manipulate or position the alternating transcutaneous current in the throughout the desired treatment depth or area.

In one embodiment, the area encompassed by the current injected, such as its cross-sectional area, is large near the electrode, relative to the cross-section flowing along a current path deeper in the tissue. As a result, near the electrode, less heating occurs because the current spreads over a large area relative to the smaller cross-sectional area of a current path in a treatment region. The injected current can be constricted to encompass a smaller area in one embodiment and thus generate more heat in the smaller area.

### BRIEF DESCRIPTION OF THE FIGURES

The foregoing and other features and advantages of the present invention will be more fully understood from the following detailed description of illustrative embodiments, taken in conjunction with the accompanying drawings in which:

FIG. 1A is schematic diagram of an electrical energy delivery system according to an illustrative embodiment of the disclosure.

FIG. 1B is schematic diagram of an electrical energy delivery system having a cuff or table or dual layer configuration according to an illustrative embodiment of the disclosure.

FIG. 1C is schematic diagram of an electrical energy delivery system that includes a handheld device and control, display, and interface devices according to an illustrative embodiment of the disclosure.

FIGS. 1D-1H are cross-sectional views of a material such as tissue in which an alternating current has been injected below the surface of the material (top of each view) and manipulated using one or more magnetic fields or static attractors according to an illustrative embodiment of the disclosure.

FIG. 2A is a plot of power versus impedance depicting a power wave form associated with an electrosurgical current.

FIGS. 2B and 2C are plots depicting a relationship between tissue impedance versus time and power versus time, according to illustrative embodiments of the disclosure.

FIGS. 3A-3C are schematic diagrams depicting cross sections of a tissue sample in which an alternating current has been generated according to illustrative embodiments of the disclosure.

FIGS. 4A-4C are schematic diagrams depicting cross sections of a tissue sample in which an alternating current has been generated according to illustrative embodiments of the disclosure.

FIGS. 5A-5C are schematic diagrams depicting various coils used to generate magnetic fields in response to an alternating magnetic field inducing current suitable for repelling an alternating current in a material according to illustrative embodiments of the disclosure.

FIG. 6 is a plot of power versus impedance and voltage versus impedance with respect to one or more power control characteristics according to an illustrative embodiment of the disclosure.

FIGS. 7A and 7B are plots of voltage versus time for a current delivery device for different operating modes according to according to illustrative embodiments of the disclosure.

FIGS. 8A and 8B are schematic diagrams depicting different arrangements of one or more magnetic field sources according to illustrative embodiments of the disclosure.

FIG. 9A is a schematic diagram depicting a side view of three magnetic coils of varying magnetic field strengths according to an illustrative embodiment of the disclosure.

FIG. 9B is a schematic diagram depicting a side view of a similar magnetic source implementation to that of FIG. 9A showing a matrix of tissue depths (A1-A3, B1-B3, and C1-C3) with varying magnetic fields according to an illustrative embodiment of the disclosure.

FIG. 9C is a schematic diagram depicting a top view of magnetic filed generating coils A, B, and C and a pair of electrodes of a electrical energy delivery system according to an illustrative embodiment of the disclosure.

FIGS. 10A-10B are schematic diagrams depicting an electrical energy delivery system that includes a plurality of magnetic field sources and static attractors according to illustrative embodiments of the disclosure.

FIG. 11 is a schematic diagram depicting an electrical energy delivery system that includes a plurality of magnetic field sources, associated drivers, a phase monitor and a control system according to an illustrative embodiment of the disclosure.

### DETAILED DESCRIPTION

The invention will be more completely understood through the following detailed description, which should be read in conjunction with the attached drawings. Detailed embodiments are disclosed herein, however, it is to be understood that the disclosed embodiments are merely exemplary. Therefore, specific functional details disclosed herein are not to be interpreted as limiting, but merely as a basis for the claims and as a representative basis for teaching one skilled in the art to variously employ the invention in virtually any appropriately detailed embodiment.

Embodiments of the invention relate to systems, methods and devices suitable for generating an alternating current (AC) in a material such as a tissue and controlling the current to adjust the path of its flow. The generated current includes flowing charge carriers that cause resistive heating or other chemical changes when the current/electrical energy is directed through various materials such as tissue, cells, medicaments and others. An injection current generated from an AC source passes from an electrode to penetrate the surface of the skin in one embodiment. The current flows as an alternating transcutaneous current from the AC source above the skin to a plurality of locations below the skin in one embodiment. The transport of charge carriers over time below the skin occurs along one or more current channels.

These current channels or paths can be moved in a controlled manner to target various regions of tissue or other materials. Current repelling and attracting sources, such as electromagnetics, can be used to move the current channel to a particular tissue region such as a region below the skin. Suitable types of tissue or material suitable for use with the devices described herein can include, without limitation, fat, collagen, blood, bone, organ tissue, water, damaged tissue, nerve tissue, cancerous tissue, and other tissue and cell types suitable for treatment using heat or current.

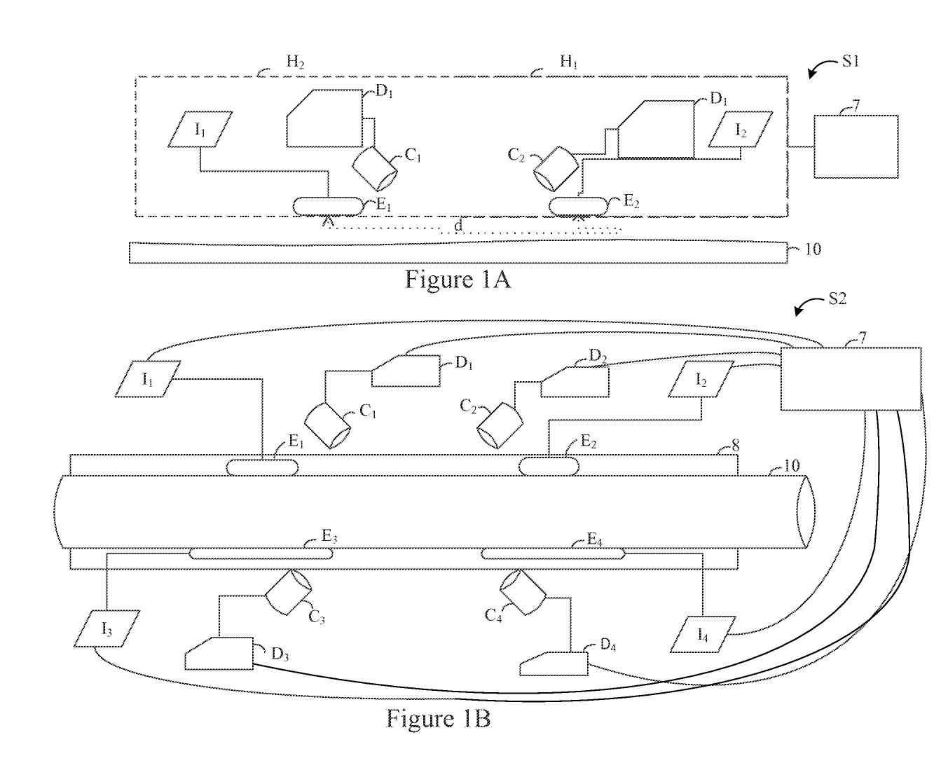

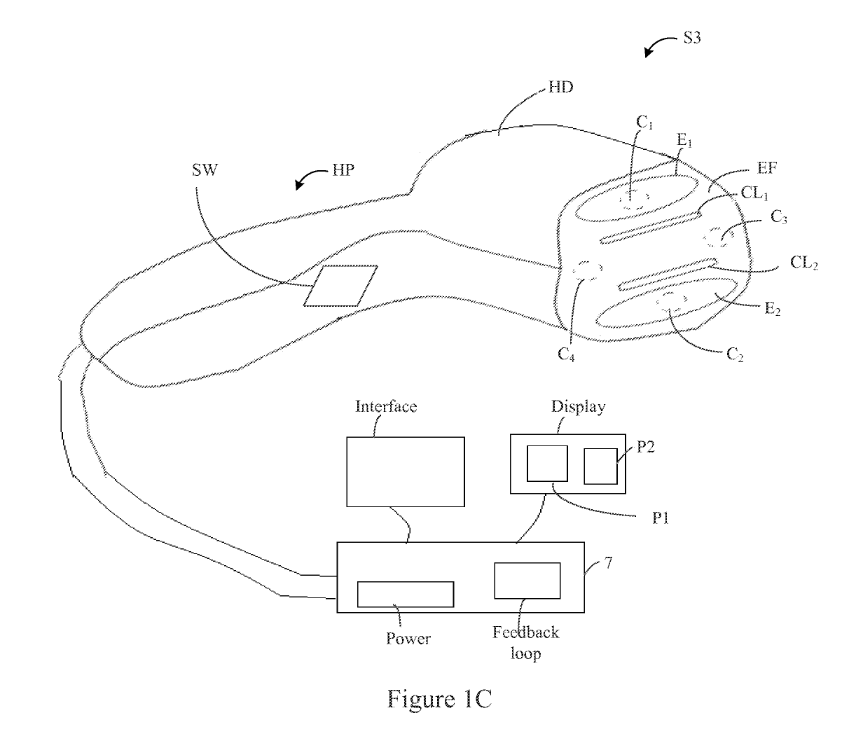

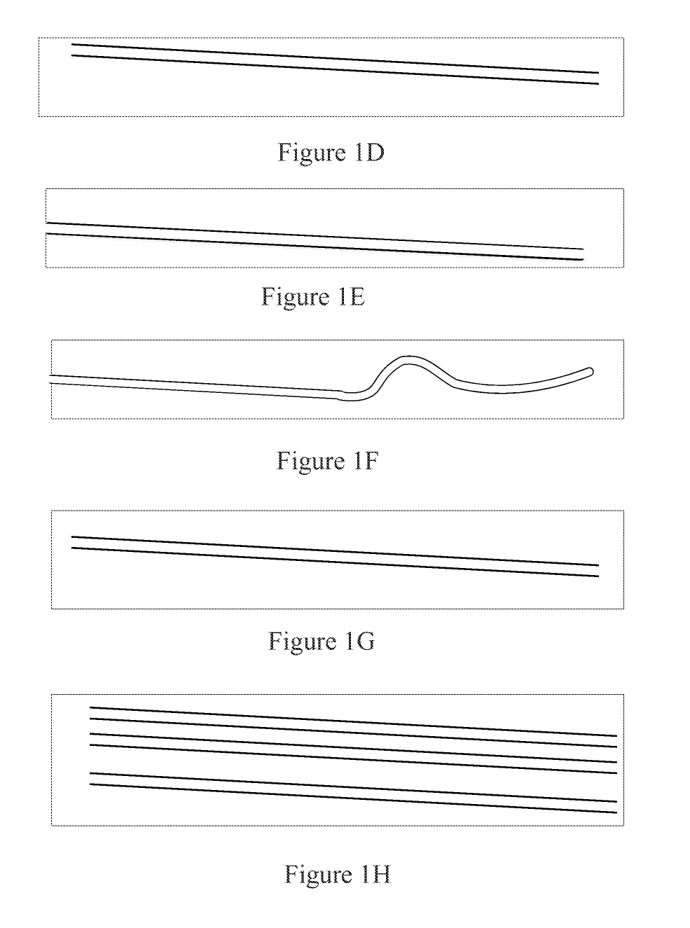

FIG. 1A shows an exemplary system S1 suitable for generating a current channel or path in a material sample 10 such as a tissue sample having a target treatment area below the sample material's surface (e.g., at a depth from a tissue surface). The various components shown can be included in individual housings designated by the dotted lines and H1 and H2 as part of S1 with the components shown in FIG. 1A disposed within a respective housing H1 and H2. The two housings H1, H2 can be a single common housing in one embodiment. In another embodiment, housing, H1 and/or H2, are not present. Housings H1 and H2 can be connected by a joint or other coupling mechanism so they can bend to grip tissues folds and other sample materials for current delivery. The housing can be of various shapes such as a cuff or table or handpiece or treatment head with a sample 10 contacting surface as described herein.

System S1 can include a control system 7 that can include an interface for controlling electrical parameters used to generate and position a current in sample 10. The control system can be in electrical communication with the various elements shown in FIG. 1A via a wire or wireless connection. In one embodiment, the system S1 includes a first electrode E1 in electrical communication with a current source I1 such as an alternating current source. In one embodiment, the system S1 includes a second electrode E2 is in electrical communication with a current source I2 such as an alternating current source. In one embodiment, current sources I1 and I2 are closed loop current sources. The housings H1, H2 can include openings in their surfaces by which the electrodes E1 and E2 can contact sample 10. In one embodiment, as shown by the dotted line with dual arrows, the distance d between the electrodes E1 and E2 can range from about 10 mm to about 100 mm. In one embodiment, the AC currents generated in a sample material 10 range from about 50 mA to about 5 A.

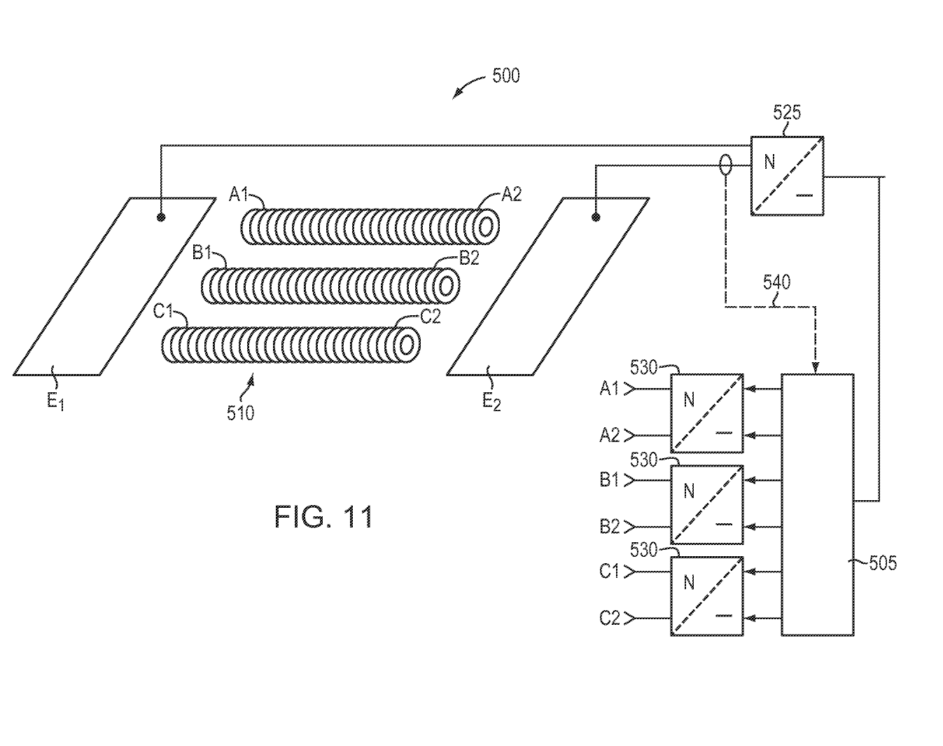

A current channel can be generated between electrodes E1 and E2 at a distance in sample 10 that can be set and changed using a control system 7. The control system 7 can include a power supply or be in electrical communication with one. The control system can include a feedback loop responsive to signals from one or more of the electrical components shown. In addition, a phase monitor that tracks phase signal changes in one or more of the electrical currents used by or generated by system S1 can be a component of the control system 7. Additional control system details are described with respect to FIG. 11 and elsewhere herein.

System S1 can include one or more magnetic field sources such as the two sources C1, C2 shown. The magnetic field sources can be implemented using coils that are driven with an alternating field inducing current. Alternating current drivers D1, D2 can be in electrical communication with each magnetic field source C1, C2, respectively. The AC current drivers D1, D2 include current sources that provide the field inducing current to each source C1, C2. The current channel can be moved in the sample material 10 by using attractive or repulsive fields generated by C1, C2 or other devices such as static attractors. The magnetic field sources C1, C2 can be oriented to direct magnetic field lines at an angle relative to the surface of material 10 or perpendicular to the surface. In one embodiment, the angle ranges from greater than about 0 to about 45 degrees.

In one embodiment, the electrodes have a curved shape to expand an injection current received from current sources I1, I2 that avoids hot spots and other thermal or electrical damage to material 10. The electrodes E1, E2 can include a buffer zone of material around them having a thickness of a fraction of d such that the buffer material from E1 can be positioned to contact E2 and set a working distance d automatically when the electrodes are affixed to the sample material 10, which is typically skin or another tissue.

FIG. 1B shows an exemplary system S2 suitable for generating a current channel or path in a material sample 10 such as a tissue sample having a target treatment area below the sample material's surface. System S2 includes the electrodes (E1, E2), current sources (I1, I2), magnetic field sources (C1, C2), drivers (D1, D2) and control system 7 of system S1. System S2 also includes a second set of electrodes (E3, E4), current sources (I3, I4), magnetic field sources (C3, C4), and drivers (D3, D4) arranged relative to sample 10 such that they are disposed across from their counterpart components from system S1.

System S2 includes a support 8 that can be a table upon which sample 10 is disposed or a cuff or tube in which sample 10 is inserted. Thus, for example, a person could lie on a table 8 or insert their arm, leg, or torso in a tube or cuff 8 and have their skin contact electrodes E1, E2, E3 and E4 as shown. The system S2 allows current to be generated in material 10 and steered or otherwise positioned from either surface or side of the material 10 using the components shown and as described herein.

FIG. 1C shows an exemplary system S3 suitable for generating a current channel or path in a material sample such as a tissue sample having a target treatment area below the sample material's surface. According to one embodiment, a current/energy delivery apparatus HP can include a hand piece that includes a head HD and an endface EF. The head HD can include two or more electrodes E1, E2 that include skin surface contacting regions such as conductive materials and a current source, such as I1 and I2 of FIGS. 1A and 1B (not shown) for non-invasively directing an alternating current through the skin's surface. The distance between the electrodes is d in one embodiment. The head HD can include or be in electrical communication with a control system 7. The current delivery apparatus HP can include a local on and off or control switch SW. In one embodiment, the distance d between the electrodes E1 and E2 can range from about 10 mm to about 100 mm.

Further, the endface EF, in addition to electrodes E1, E2, can include magnetic field sources C1, C2, C3, and C4 disposed in the housing of the apparatus HP. Although four sources are shown, one or more sources can be used in a given embodiment. The arrangement of the magnetic field sources C1, C2, C3, and C4 is typically around the region within or bordering around the two electrodes E1, E2. In this way, the magnetic field sources C1, C2, C3, and C4 can be used to push a current generated between E1 and E2 deeper into a sample material and be maintained at a location or move around within desired locations in the material. For example, the magnetic field sources C1, C2, C3, and C4 can be used to push a current generated at the tissue surface into a depth of tissue such as between E1 and E2 that is deeper into a tissue. Once pushed below the skin surface, the current can be maintained at a location or moved around within desired locations in the material. In this way, targeted subsurface current-based treatment can be performed.

According to one embodiment, a current range from about 100 mA to about 1 A is used. This alternating current is generated and expanded through a first electrode and uniformly injected through the patient's skin surface. The expanded injection surface area minimizes energy densities and avoids significant surface damage. The current is directed through a sample surface such as the skin surface from the first electrode E1 toward a second electrode E2 having an opposite polarity from the first electrode E1. The current passes through regions where externally applied magnetic fields are applied using one or more of magnetic field sources C1, C2, C3, and C4, which pushes a transcutaneous current away from the surface of the skin and deeper into target tissue regions such as fatty adipose tissues.

As tissue is treated by currents generated using systems S1, S2, S3 and other devices, systems and methods described herein, the tissue impedance changes over time. This occurs as a result of resistive heating of the tissue. As the tissue heats and impedance changes, current flows out of the primary treatment area to unheated areas. In one embodiment, the HP of FIG. 1C includes localized cooling elements CL1 and CL2 as shown. These can be used to reduce impedance effects and to cool the skin to prevent damage in the vicinity of electrodes E1, E2.

The current generated in the tissue or other sample material results from the applied voltage through the changing tissue impedance. Operating voltages used in the systems S1, S2, S3 to generate a current in the tissue are typically below about a peak voltage of 250V. The control system 7 includes a feedback loop to actively manage the systems S1, S2, and S3 using a closed loop. The control system can include a supervisory power limiter rather than a direct power (wattage) control approach commonly employed in invasive electrosurgical systems. The control system can be connected to a display that includes a plurality of panels shown by exemplary panels P1, P2. These panels can display current, voltage, temperature, images, and other information of interest when using a given current generating system or method. An interface for controlling the HP or other embodiment can also be part of or connected to control system 7. The control system 7 can include a power supply and a feedback loop.

FIG. 1D through FIG. 1H show various electrical current channels from a cross-sectional perspective and how they change and are modified using the methods and devices described herein. FIG. 1D show an initial current channel before it is subjected to a magnetic field to push it down lower into a sample as depicted in FIG. 1E. In FIG. 1F, a current channel that includes a ripple is shown. This ripple or other non-linear portions of current channel can be substantially linearized, as shown in FIG. 1G, using the magnetic field sources alone or with an attractor used to pull the current channel in a first direction while the magnetic field sources push it in another direction. Various current channels and portions thereof can be further manipulated using magnetic fields to form an open loop (e.g., shaped like the letter “C”) or a closed loop (e.g., shaped like the letter “O”). Finally, in FIG. 1H various current paths that change over time are displayed simultaneously. Thus, as shown in FIG. 1H, a current path can be migrated up and down within a plane or a volume in material 10 to effectively scan a region of tissue and heat it in a substantially uniform manner.

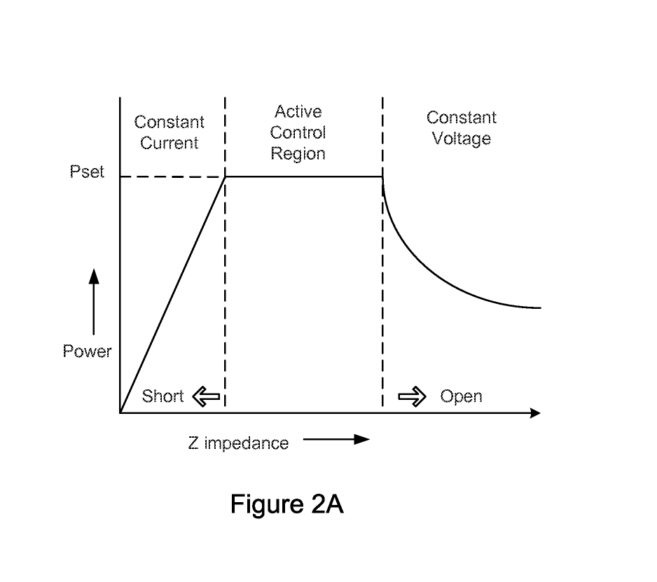

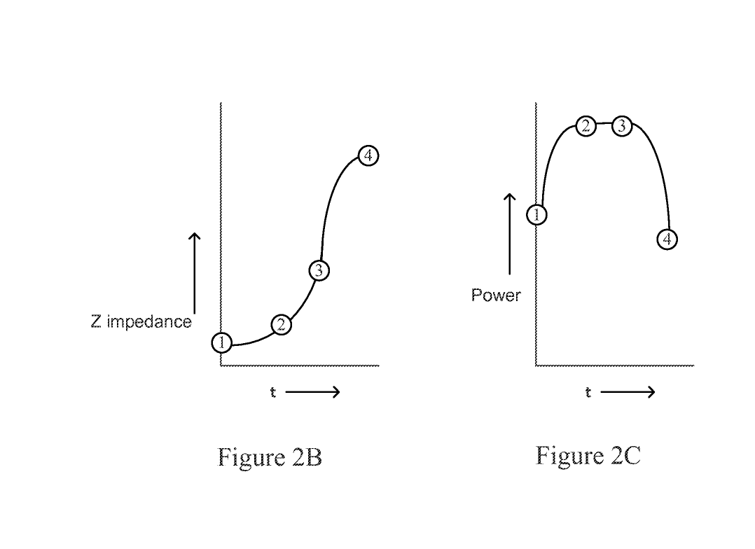

Prior to considering some additional details and embodiments, it is useful to consider some of the impedance relationships that result in current generated in a sample moving in an uncontrolled manner. FIG. 2A depicts a typical electrosurgical power wave form as a function of impedance, Z. As the power increases initially under a constant current or short circuit period, the impedance Z increases linearly until a predetermined power level, P_{SET} is achieved. The power output remains constant during an active control region as the impedance Z increases until a constant voltage is achieved. After passing through the active control region, the power output begins to decline as the impedance continues to increase. It is during the constant voltage, or open circuit, period, the current begins to deviate from its initial path and flow out of the primary treatment area of the tissue. This problem can be addressed using the current channel directing components of systems S1, S2, and S3 and as otherwise described herein.

FIGS. 2B and 2C depict plots of the tissue impedance and power output, respectively as a function of time, each plot shows corresponding points in time 1 to 4. As shown in FIG. 2B, the impedance of the tissue during treatment increases over time (point 1 to 4). Power output, as shown in FIG. 2C increases for an initial period (point 1 to 2) before reaching a plateau. After a period of time, (point 2 to 3), power output significantly declines (point 3-4). The decline in power output is attributed to the increased impedance (points 3-4).

According to one embodiment of the disclosure, power losses in treatment region caused by and associated/redirected current flow may be reduced by trapping the current flow within the treatment area. A magnetic field can be used to constrain current flow to a cooler tissue region. The injected current path remains trapped in the treatment zone despite the tissue impedance changes. The systems S1, S2, and S3 and others described herein facilitate steering or trapping a current channel within a treatment region of interest.

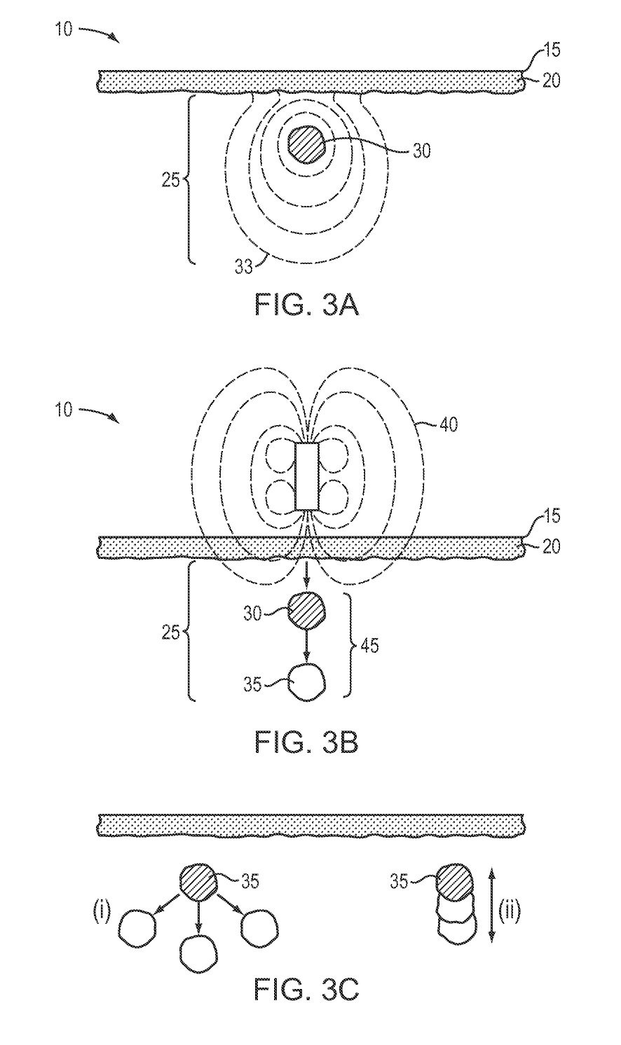

FIGS. 3A-3C depict cross-sections of a tissue sample 10 that includes a skin layer in which a current has been generated using embodiments of the disclosure. FIG. 3A depicts a cross-section of a sample 10 having a current 30 applied through the tissue prior to the use of an external magnetic-field. The sample includes, for example, an area of skin and one or more layers such as the epidermis 15 and the dermis 20 disposed above a target tissue layer 25 to be treated. The current flow 30 cross-section represents the preponderance of the high density current flow traveling through the tissue. Under normal electrosurgical current applications, the high density current 30 flow will move about the tissue as the impedance of the tissue increases. The current flow will also expand out from a central transport path or channel, shown by concentric circles 33 as the impedance increases.

FIG. 3B depicts current flow cross-section 30 without a current attracting or repelling field and a modified current flow cross-section 35 under the application of a magnetic field 40. As shown, current flow 30 travels through the tissue sample at a first depth. Upon application of the magnetic field 40, a current offset distance 45 is created by the interaction of the magnetic field lines, or flux, with the electron flow of the current cross-section 30. The field 40 pushes current flow 35 away from the field 40 and deeper into the tissue sample 25 an offset distance 45.

According to one embodiment, as depicted in FIG. 3C, an applied magnetic field, such as from a magnetic or electromagnet, changes a flow path for the current flow 35. Depending on the direction, position, and intensity of the magnetic field, i.e., angle of application, the current flow path can be repositioned laterally, as well as longitudinally, as shown in FIG. 3C(i) by the three transitional positions. In another embodiment, as shown in FIG. 3C(ii) the magnetic field may be applied using oscillating frequencies from the current source to move the current channel back and forth or another type of periodic motion. Accordingly, by varying magnetic field strength, the energy delivered to a tissue region through a current flowing along a current channel is enhanced by scanning the current channel over the tissue region.

In one embodiment, an exemplary current delivery apparatus includes at least one externally applied magnetic field used to position a current channel below the surface of the skin. According to one embodiment, a current delivery device includes an adjustable RF injection current source and at least two electrodes. One or more electromagnetics that include a coil can also be used to direct or expand the current channel. These devices allow the injection current to penetrate a sample and cause uniform heating without creating hotspots.

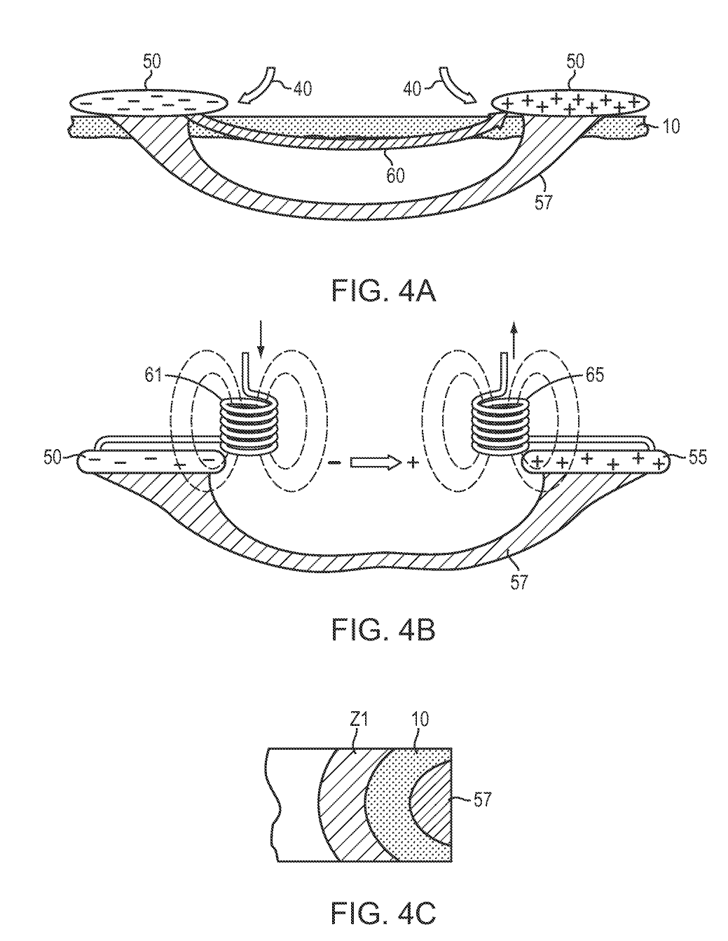

FIGS. 4A-B depicts an implementation of one embodiment of the disclosure. The illustrative device includes at least one externally applied magnetic field source. This source steers or directs the injected current as it propagates through a path in a sample flow direction. The magnetic field comprises field lines that apply a displacing force that migrates a current channel deeper into the patient's tissue, away from the epidermis and into regions of the dermis and fat layers.

As shown in FIG. 4A, a negative electrode 50 electrically connected to a current source (not shown) may be placed on the skin surface near the treatment area. A positive electrode, 55 may be placed on an opposing side of the treatment area. An alternating current flows to the negative electrode 50 from the current source. The current travels within the tissue along a path towards the positive electrode 55. The current then flows out of the tissue and skin and to the positive electrode 55.

FIG. 4A depicts a first current flow 60 that travels shallowly along the underside or through the upper layers of the skin while not reaching any substantial depth of the tissue to be treated. The initial current channel or path 60 fails to penetrate into regions of tissue disposed further below the skin's surface. When a magnetic field 40 is applied, in accordance with one embodiment of the disclosure, a modified current channel or path 57 is formed at a depth below that of the initial current channel 60 and penetrates deeper into the tissue such that it can reach a target treatment region. Redirecting a current channel to a deeper location can reduce damaging effects on skin and improve efficacy of tissue treatment.

FIG. 4B depicts another embodiment of the disclosure in which two magnetic fields may be applied at or near each of the electrodes 50, 55. A magnetic field 61 at the negative electrode 50 is created by a current passing from the current source through a magnetic coil 61 and then to the electrode and into the tissue sample. Similarly, a positive electrode 55 is connected to a source by a positive magnetic coil 65. The embodiment of FIG. 4B may use a single current driven from the negative electrode to the positive electrode, or alternatively, each electrode can be driven separately, allowing for more precise electrode conductivity control. FIG. 4C shows a top-down view around one an electrode 55, in which adjacent bands of impedance zones Z1 surround the skin 10 and injected current channel 57 as a result of the proximity of the adjacent magnetic coil 65.

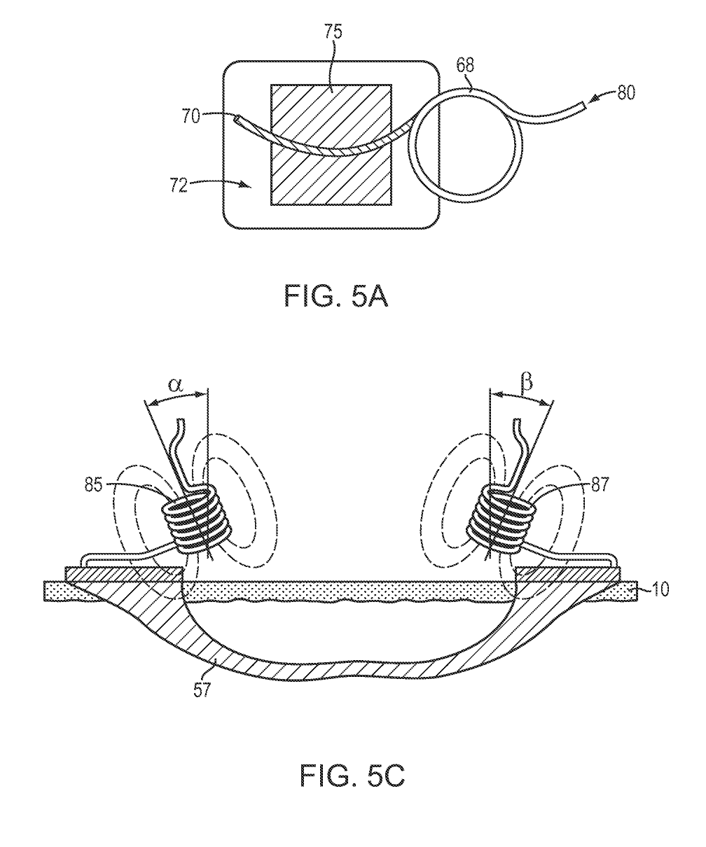

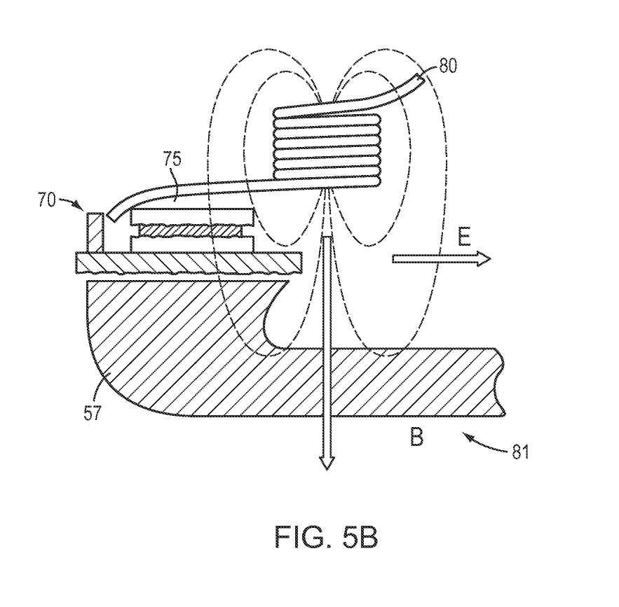

Turning now to FIGS. 5A-5C, various embodiments of a current/energy delivery device are depicted. FIG. 5A depicts a top-down view of an electrode input terminal 70 including a negative electrode 72. The device can further includes a cooler such as a thermo electric cooler (TEC) 75 and an injection coil 80. The TEC 75 serves to keep the temperature of the skin underneath the electrode input terminal low enough to avoid burning or other dermal damage. FIG. 5B depicts a side-view of the embodiment shown in FIG. 5A with the electric field E and the magnetic field B at right angles to each other relative to coil 80. The current channel 57 flows below the lines of magnetic flux from the coil 80. The injection coil 80 is arranged such that upon the application of the injection current, the magnetic field B created by the coil faces is directed along a longitudinal axis of the coil toward the skin's surface 10.

FIG. 5C depicts a side-view of another embodiment of the disclosure in which two injection coils are used to generate a controlled current flow at a target region below the skin surface. The inward angling of the negative coil 85 and the positive coil 87 increase the magnetic field effect on the electrode by increasing the density of field lines in regions of interest above a target treatment region. The application of the two fields pushes the current flow deeper into the tissue in the areas proximate to the electrodes. The center portion of the current flow begins to rise near the center of the treatment area where the magnetic fields are weaker than at the electrode contact areas. The coils need not be in series, in parallel or otherwise in electrical communication with the current sources. Instead, each of the current sources, static attractors, and magnetic field sources can all be electrically isolated from each other. In one embodiment, each of the foregoing is in electrical communication with a control system and a power supply. Each coil can be oriented at an angle relative to a normal to the skin surface such as angles β and α as shown. In one embodiment, these angles ranges from about 5 degrees to about 45 degrees. The angles β and α can be substantially the same or different in a given implementation for current channel placement in a tissue volume.

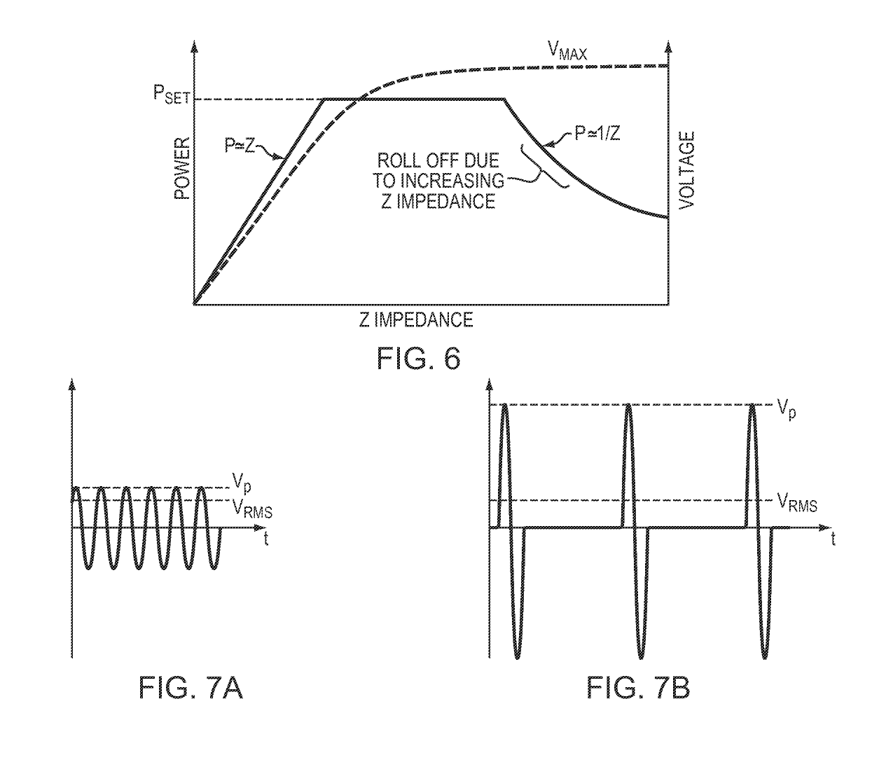

FIG. 6 depicts power control characteristics of a current/energy device in accordance with one embodiment of the disclosure. Power (left axis) and voltage (right axis) are shown as a function of impedance. Both the power and impedance increase on a near one-to-one basis as higher voltages are achieved. Accordingly, power is approximately equal to impedance during the first linear portion of the power function. As the voltage nears its maximum, V_{MAX}, the power reaches the P_{SET}. When V_{MAX} is reached, the power output experiences a significant roll off inversely proportional to the impedance, which is still increasing.

FIGS. 7A and 7B are plot of a current/energy device's voltage application of a low-voltage, pure cut mode and a high voltage, coagulate mode, respectively, as a function of time. According to traditional electrosurgical models, the pure cut mode of FIG. 7A utilizes an oscillating voltage Vₚ of 500V, a V_{RMS} of 354V, and a continuous duty cycle. The pure cut mode has a crest factor of 1.4. In the pure coagulate mode, shown in FIG. 7B, a high voltage oscillating voltage Vₚ reaches 1.77 kV and a duty cycle of about 20% and a crest factor of 5. High voltage application can lead to current arcing which further increases the temperature of the skin surfaces. Therefore, to reduce arcing, maintaining a low peak voltage, Vₚ to minimize cauterization is advantageous. This may be achieved by implementing a quasi-cut mode in which Vₚ is kept low, and a high duty cycle to limit tissue damage that might be caused by arcing. In one embodiment, Vₚ ranges from about 100 Vpk to about 350 Vpk when operating one of the energy/current deliver devices.

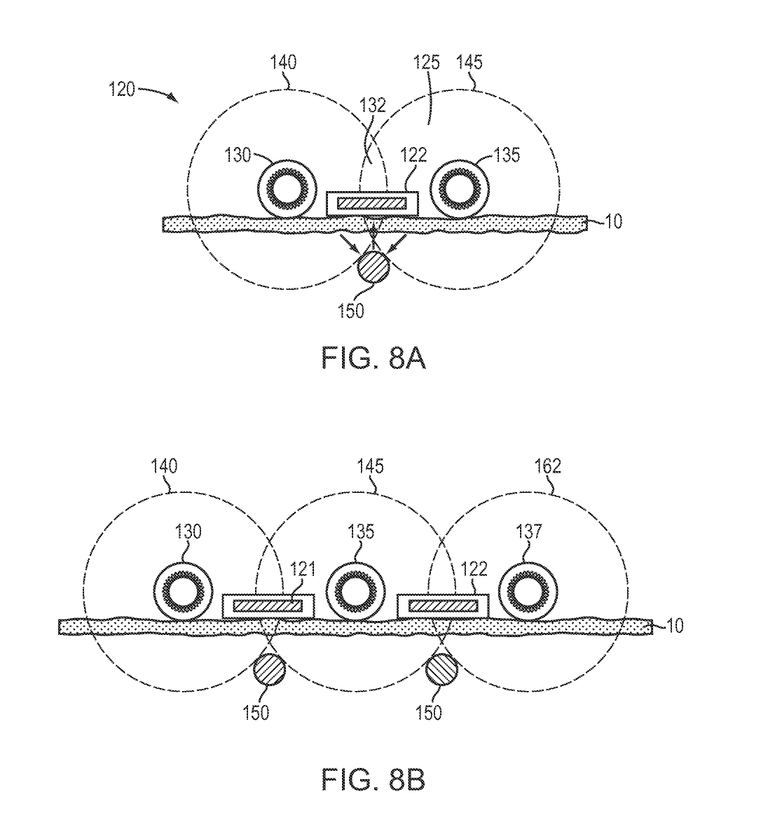

According to another device embodiment 120, depicted in FIGS. 8A and 8B, one or more insulated static attractors 121, 122 and/or magnetic coils 130, 135, 137 may be arranged parallel to or otherwise disposed on or near the skin surface. The insulated static attractors 121, 122 are used to provide flux cancelling mirror currents on insulated conductor coil to attract the current flow 150 while the magnetic fields of the coils 130, 135, 137 push the current flow deeper. The outer boundary of the magnetic field is shown by dotted regions 140, 145, 162.

The attractor and coil arrangement of FIG. 8B substantially linearizes current flow along a current channel or otherwise provides lateral stabilization of the current flow. That is, the current can be effectively linearized in a target region of tissue using attractors and magnetic field sources to selectively push and pull along a current channel to promote a substantially linear path. The current channel includes one or more portions or sections that are line segments rather than ripples for the majority of the current channel's length in one embodiment. The current flow, while being drawn towards the static attractor, is simultaneously bounded by two magnetic fields emanating from the coils on the surface. According to one embodiment, as shown in FIG. 8B, three magnetic coils 130, 135, 137 and two static attractors 121, 122 are arranged such that multiple current paths can be modified to facilitate current delivery to deeper tissue depths. A region 132 of an intersection of field lines above an attractor is also shown.

According to another embodiment of the disclosure, a plurality of magnetic field sources, such as coils with alternating field inducing current passing through them, of varying intensity can be positioned by a user or disposed in a hand piece, cuff, table, or other configuration to direct an alternating current to a target treatment region. Use of strategically placed magnetic field sources can create free travel zones for the electrons of the application current. One or more static attractors can also be used in various embodiments to help shape and direct the current channel.

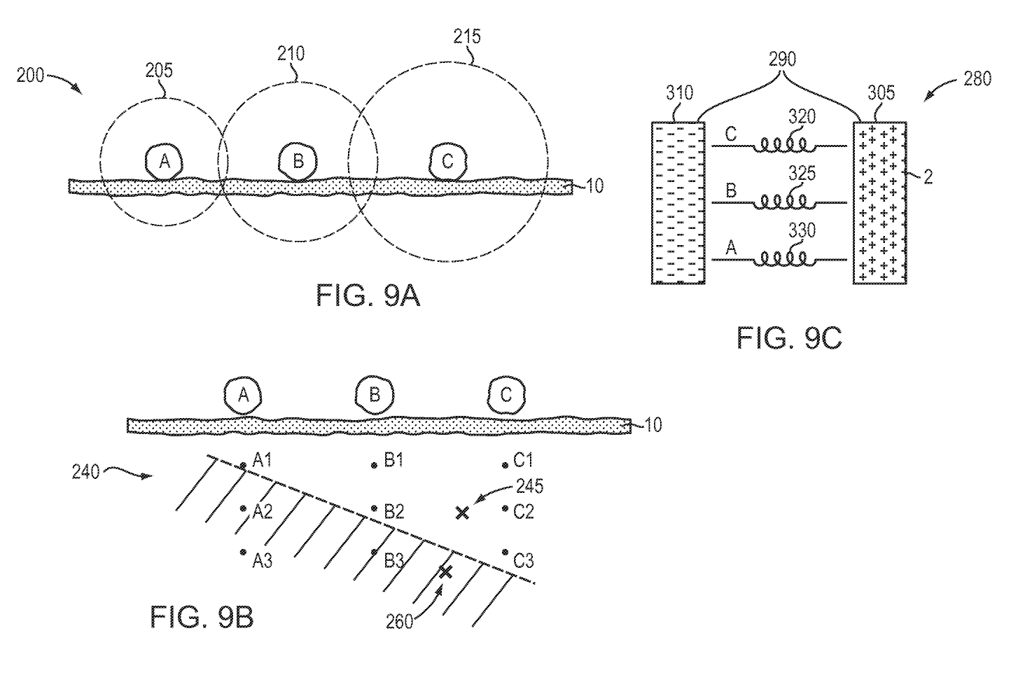

FIG. 9A depicts a side view of a current delivery system 200 that includes three magnetic coils A, B, C of varying field strengths 205, 210, 215. Coil A producing a small magnetic field 205, coil B producing a medium field 210 and coil C producing a larger field 215. FIG. 9B depicts a side view of a similar implementation of a system 240 showing a matrix of tissue depths below each of coils A, B and C (A1-A3, B1-B3, and C1-C3) with the varying magnetic fields. The illustrative embodiment shown in FIG. 9B depicts a sloping tissue treatment boundary extending from A1 to B2 to C3. The magnetic field strengths in FIG. 9B are the same as FIG. 9A and increase from left to right. Points above the treatment boundary (i.e., B1, B2, C1, C2, C3) are unaffected by the current flow, with point 245 being in the current exclusion zone, while the areas in the treatment area (i.e., A1, A2, B3) receive the highest concentrations of current flow as bounded by the magnetic fields emanating from the three coils, with point 260 being in the electron free travel zone. FIG. 9C depicts a top-down view of the illustrative device including coils A, B, and C, also identified as 330, 325, 320 between an electrode pair 290. The electrode pair 290 includes a negative electrode 310 and a positive electrode 305.

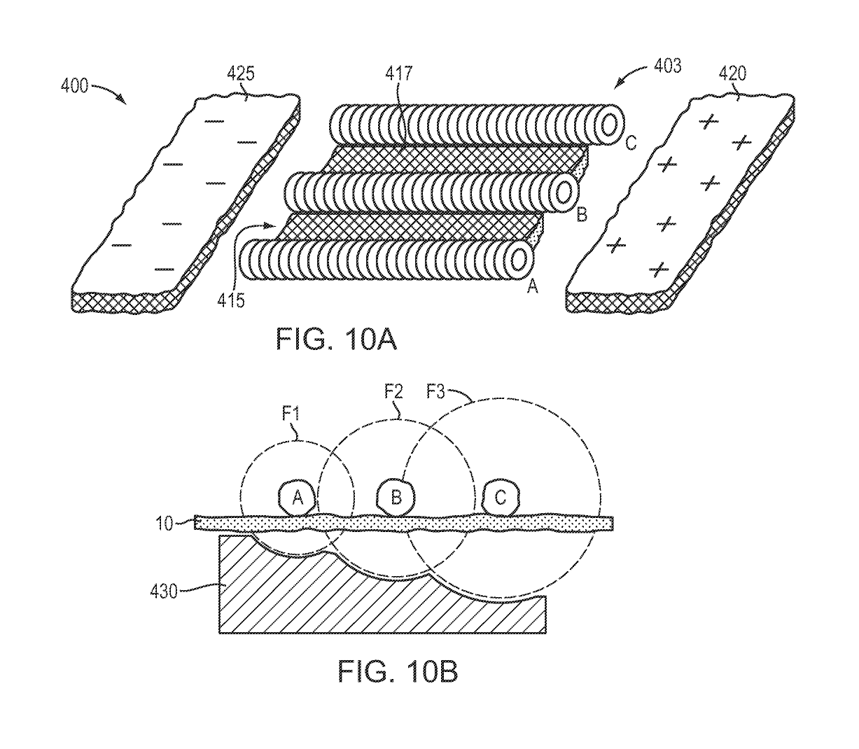

FIGS. 10A-10B depict a device embodiment 400 of the disclosure utilizing multiple parallel coils and static attractors. FIG. 10A depicts a perspective view of a device 400 in accordance with an embodiment of the disclosure including magnetic coils A, B, and C, two static attractors 415, 417 and a negative 425 and positive 420 electrode. FIG. 10B depicts a side-view where magnetic fields of increasing strength are applied to create a shaped treatment area. According to the illustrative embodiment, coil A outputs a field F1 using a 10 A current, coil B outputs a field F2 using 20 A current and coil C outputs a field F3 40 A current. The varying strength of the fields F1, F2, and F3 creates a free travel zone for electrons 430 and creates a broader area of treatment. The creation of electron travel and exclusion zones using attractors and coils can improve targeted current delivery and thus reduce the number of applications and potential tissue damage arising from over excitation of the skin and tissue. The electron free travel zone 430, shown by the hashed lines, is formed, in part, by the portions of the circumferences of the fields F1, F2, and F3. This zone 430 can be shaped to allow various current channel geometries and locations within the zone 430 for tissue treatment via current delivery/heating.

The embodiments described herein can include various control systems. An exemplary current/energy delivery system 500 is depicted in FIG. 11. The system 500 includes a control system 505. As discussed herein, a first electrode E1 and a second electrode E2 are disposed on the skin surface or another suitable material. A plurality of magnetic field sources 510 shown as three electromagnetics is disposed between the electrodes E1, E2. Each of the electromagnetic includes a coil having a first end and a second end, which are labeled A1, A2, B1, B2, and C1, C2, respectively. The control system 505, which can be system 7 described herein or a component thereof, is in electrical communication with electrode E1 as shown. Each of the electrodes E1, E2 is in electrical communication with AC electrode driver 525. The electrode driver 525 includes a closed loop current source. In one embodiment, the magnitude of the current is adjustable by a user through the control system 505 or an interface device in communication with the control system 505.

The plurality of electromagnets 510 are also each in electrical communication at each of their respective ends A1, A2, B1, B2, and C1, C2 with a magnetic field driver 530 as shown. Each of the magnetic field drivers 530 control each electromagnet on an individual basis using system 505. This individual control scheme allows both a magnitude and a phase angle to be set for the alternating field inducing current used to drive each of the plurality of electromagnetics 510. A phase monitor 540 is included in the system 500 and allows the phase of the electrode current to be relayed to the control system 505. This phase value can be used to automatically match the phase of the AC signal for one or more of the electromagnets 510. In one embodiment, all of the magnetic field source AC drivers 520 are “in phase” with the electrode current. In another embodiment, a predetermined control system phase deviation can be specified that deviates from the “in phase” scenario described herein. In one embodiment, the electrode and magnetic field source drivers are all closed loop AC current sources

In one embodiment of the disclosure, as the treatment progresses, more amp-seconds are delivered and the treated tissue temperature increases resulting in an impedance increase. As the impedance increases, the injected treatment current tends to wander outside the desired treatment zone due to the adjacent areas remaining untreated and therefore of a lower impedance. Correspondingly, as the impedance rises greater intensity magnetic fields are required to hold the treatment current within the desired treatment zone. Thus, an upper limit for treatment tissue impedance occurs at the boundary where the applied positioning magnetic fields are no longer able to keep the treatment current in the desired position.

Similarly, a limit exists as to the amount of impedance change permitted in tissue because this correlates with the temperature rise of the treated volume of tissue. In one embodiment, the control system regulates one or more of treatment time, current delivered, amount of cooling, and other parameters described herein in response to a threshold being met or exceeded with respect to an amount of impedance changes in a tissue, temperature of the tissue, an impedance value or magnetic field value. In one embodiment, a temperature sensor or an impedance sensor is used to measure tissue temperature or tissue impedance, respectively, as an input to the control system to determine if a threshold has been exceeded or met.

The systems, devices and methods described herein are suitable for directing current into tissue to a target region in spite of the current directing issues caused by impedance changes resulting from resistive heating. The devices and systems can direct current into areas of fatty tissue to promote fat loss through tissue heating and targeted tissue damage or ablation. Further, skin tightening (laxity improvements) or small vessel vascular treatments can also be facilitated by remodeling tissue under the skin or by specifically targeting small vessels. Subcutaneous tissue ablation can also be performed using the non-invasive techniques described herein. Targeted tissue heating to stimulate medicament update or healing of damaged tissue can also be performed using the systems and device described herein.

Advantages of certain embodiments include a device that is carries a high inherent safety factor, simplicity of use, and a low equipment and operations cost, as compared to lasers for example. Further, embodiments of the disclosure provide a unique combination of an electrosurgical device and external magnetic fields, which allow the noninvasive manipulation of invasive treatment currents. Additionally, embodiments of the disclosure are flexible to many variations in frequency, voltage, current and geometry of construction.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

While the invention has been described with reference to illustrative embodiments, it will be understood by those skilled in the art that various other changes, omissions and/or additions may be made and substantial equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. An electrical energy delivery apparatus comprising:
a control system comprising a user interface;
an alternating current source comprising a current output, the alternating current source in electrical communication with the control system;
a first electrode in electrical communication with the current output;
a second electrode disposed a distance d from the first electrode; and
a first magnetic field source comprising a first magnetic field output, the first magnetic field source in electrical communication with the control system, the first magnetic field source disposed between the first electrode and the second electrode.

2. The electrical energy delivery system of claim 1, wherein the alternating current source is a closed loop current source.

3. The electrical energy delivery system of claim 1, further comprising a first housing comprising a first housing surface defining a first opening, wherein a portion of the first electrode spans the first opening.

4. The electrical energy delivery system of claim 3, further comprising a second housing comprising a second housing surface defining a second opening, wherein a portion of the second electrode spans the second opening.

5. The electrical energy delivery system of claim 1, further comprising a housing comprising a housing surface defining a first opening and a second opening, wherein a portion of the first electrode spans the first opening, wherein a portion of the second electrode spans the second opening.

6. The electrical energy delivery system of claim 1 further comprising a second magnetic field source comprising a second magnetic field output, the second magnetic field source disposed between the first electrode and the second electrode, the second magnetic field source in electrical communication with the control system.

7. The electrical energy delivery system of claim 1, wherein d ranges from about 10 mm to about 100 mm.

8. The electrical energy delivery system of claim 1 further comprising a phase monitor in electrical communication with the control system and the second electrode.

9. The electrical energy delivery system of claim 8 wherein the control system comprises a feedback loop that receives a first phase value at the second electrode and adjusts a second phase value associated with field inducing current of the magnetic field source.

10. The electrical energy delivery system of claim 1 wherein current generated from the alternating current source ranges from about 50 mA to about 3 A.

11. The electrical energy delivery system of claim 1 further comprising a cooler in electrical communication with the control system.

12. The electrical energy delivery system of claim 1 further comprising a first driver in electrical communication with the first magnetic field source, the first driver in electrical communication with the control system, wherein the first magnetic field source comprises a first coil.

13. The electrical energy delivery system of claim 1 further comprising a static attractor in electrical communication with the control system.

14. A method of directing electrical energy to one or more locations in a region of tissue below a skin surface comprising:
generating an alternating transcutaneous current that flows between a first electrode and a second electrode to define a first current channel having a first length, the first electrode and the second electrode separated by a distance d and disposed on the skin surface;
noninvasively applying one or more magnetic fields to the skin surface that repel the alternating transcutaneous current in a direction opposite that of the skin surface until the alternating transcutaneous current reaches one or more locations and defines a second current channel having a second length, the second length greater than the first length; and
heating tissue in a target region that includes a portion of the second current channel disposed between the first electrode and the second electrode.

15. The method of claim 14 further comprising substantially linearizing the second current path such that the flow of the alternating transcutaneous current occurs along a substantially straight line segment which defines more than 50% of the second length.

16. The method of claim 14 further comprising generating the one or more magnetic fields using an alternating magnetic field inducing current passing through a coil.

17. The method of claim 16 further comprising synchronizing a first phase of the alternating transcutaneous current and a second phase of the alternating magnetic field inducing current such that the first phase and the second phase are substantially the same or offset by a predetermined control phase value.

18. The method of claim 14 wherein the one or more magnetic fields are noninvasively applied at an angle measured relative to a normal to the skin surface, wherein the angle ranges from about 5 degrees to less than or equal to about 45 degrees

19. The method of claim 14 further comprising cooling the skin surface in a region around the first electrode and the second electrode.

20. The method of claim 14 further comprising moving the alternating transcutaneous current within a tissue region back and forth between the second current path and another current path by periodically changing the applied magnetic field.

21. The method of claim 14 further comprising moving one or more sections of the second current path using one or more static attractors disposed between the first electrode and the second electrode.

22. The method of claim 14 further comprising cooling the tissue such that an impedance change results by which the transcutaneous current moves to another treatment region.
